(19) United States
(12) Patent Application Publication
(10) Pub. No.: US 2025/0281747 A1
(43) Pub. Date: Sep. 11, 2025

(54) SYSTEM, METHOD, AND APPARATUS FOR UNDERLID HOME-BASED MEIBOMIAN GLAND DISEASE TREATMENT VIA RESISTIVE HEATING AND MAGNETIC-BASED MECHANICAL STIMULATION

(71) Applicant: VERILY LIFE SCIENCES LLC, Dallas, TX (US)

(72) Inventors: Shungneng Lee, South San Francisco, CA (US); Tim Stowe, South San Francisco, CA (US)

(21) Appl. No.: 18/601,436

(22) Filed: Mar. 11, 2024

(51) Int. Cl. A61N 1/36; A61N 1/372

(52) U.S. Cl. CPC A61N 1/36046; A61N 1/36034; A61N 1/36128; A61N 1/37223

(57) ABSTRACT

A system, method, and apparatus is configured to treat MGD by applying resistive heating and magnetic-based mechanical stimulation of the eyelid. The system includes an underlid device configured to be inserted on the surface of the eye under the eyelid, and a stimulator wand configured to be held by the user on or adjacent to the eyelid in the area of the underlid device. The stimulator wand generates radio frequency (RF) energy that is used to induce an electric current in the underlid device, which is used to excite resistive heating elements that generate heat for heating the meibomian glands. The stimulator wand also generates an oscillating magnetic field that attracts ferromagnetic elements on the underlid device and induces oscillatory movement in the underlid device. The oscillatory movement of the underlid device applies a massaging motion to the meibomian glands while, at the same time, heating the glands via the heating elements.

### TECHNICAL FIELD

This disclosure relates to a system, method, and apparatus for treating meibomian gland disease/dysfunction (MGD) via resistive heating and magnetic-based mechanical stimulation.

### BACKGROUND

Meibomian gland disease/dysfunction (MGD) is upwards 80% of the root cause issue for patients with dry eye disease. Currently, there are two mildly performing drugs that treat this disorder, another extremely effective method, administered in-clinic by a machine called the LipiFlow, and another older, but also effective method using intense light pulses (ILP), which is also administered in-clinic. Other than the drugs, which have questionable effectiveness, there exists a lack of effective home-based methods for treating MGD dry eye.

The ocular surface of the eye is coated with a three-layer film commonly referred to as a tear film or tears. Referring to FIG. 1, the three layers of tear film are a mucin layer, which adheres to the ocular surface (epithelium), a middle aqueous layer, and an upper/outer meibum layer. The mucin and aqueous layers are produced by the lacrimal gland and other glands within the conjunctiva of the eye. The meibum layer is produced by meibomian glands in the eyelid. Tears flow from the ocular surface through the nasolacrimal canal, which drains into the back of the nose and throat.

Referring to FIG. 1, the mucin layer of the tear film is hydrophilic, which helps spread the aqueous layer evenly over the ocular surface. Together, the mucin and aqueous layers aid in maintaining the lubrication across the ocular surface and reducing shear stress during blinking or rubbing. Meibum forms the top layer of the tear film, helps prevent the aqueous layer from evaporating too quickly, which in turn keeps the aqueous layer free of dry spots and thereby lubricates the ocular surface for a longer period of time without needing refreshed. Tear film is anti-microbial and helps maintain the health of the eye, as well as clear any contaminants or particles that might come into contact with the eye. A more detailed cross-sectional illustration of the tear film is shown in FIG. 2.

Dry eye disease, or DED, affects millions of people worldwide. According to some studies, the third most common reason for visiting an ophthalmologist's office is for dry eye disease symptoms. Recently it has been shown that up to 80% of dry eye cases also have a component called meibomian gland dysfunction or MGD. Normally, the lipid layer produced by the meibomian glands (see FIG. 3) spreads evenly into a thin (thickness in nanometers) protective film over the air-tear interface above the cornea. This lipid can also be referred to as “meibum.” Every time a person blinks a slight amount of lipid protective film is spread. There are, however, many conditions under which this oily layer no longer spreads out evenly over the tear film and this process can be interrupted, reduced, or even stopped entirely. These root causes can include but are not limited to hormonal changes in the oil production properties with age, skin mites living in the eyelashes, prolonged infection such as difficult to remove styes, general inflammation (Blepharitis), autoimmune diseases or allergic reactions, and more recently the inadequate blinking from excessive screen time known as computer vision syndrome (CVS).

The absence of an outer protective lipid layer increases the evaporation rate of the tear film covering the eye, leading to the possibility of dry spots over the cornea epithelium. This is measured quantitatively as the so-called tear film break-up time metric abbreviated as TBUT or TFBUT.

There are two main classifications for MGD or dry-eye disease. Aqueous Deficient Dry Eye (ADDE) accounts for approximately 30% of dry-eye disease cases. Evaporative Dry-Eye (EDE) accounts for approximately 60% of dry-eye disease cases.

With ADDE, the lacrimal gland fails to produce a volume of tears sufficient to effectively coat the eye. This leads to tear hyperosmolarity, a state in which the osmolarity of the tears exceeds that of the epithelial cell, leading to reduced cell volume and increased concentration of solutes. Factors known to contribute to ADDE include the following:

| | |
|---|---|
| Aging | Refractive Surgery |
| Low Androgens | Contact Lens Usage |
| Autoimmune Disorders | Anesthesia |
| Lacrimal Obstructions | Frictional Damage |

With EDE, a deficient or unstable tear film lipid layer results in a high rate of tear film evaporation. Factors known to contribute to ADDE include the following:

| | |
|---|---|
| Vitamin A Deficiency | Contact Lens Usage |
| Preservatives from ADDE | Frictional Damage |

### Drops

In the past, mild MGD has been addressed by using warm compresses, eyelid cleansing compounds, and massaging the eyelids gently so that the meibum clogging the meibomian gland orifice is expressed or cleared. These approaches, however, have not been shown to clinically be effective in the majority of severe dry eye cases.

More recent treatments involve heating up the meibomian glands from inside the eyelids to melt or soften the meibum oils clogging the meibomian gland openings. As the eyelids are heated from the inside, heat is delivered directly to the meibomian gland. In some instances, air bladders are also used to massage the meibomian glands to help express the softening oil clogs from the glands. However, such procedures can still be highly invasive and costly procedures requiring an expert ophthalmologist, anesthesia, and multiple treatments each year.

Other known eye treatments include heating the outside of the eyelids using heating pads. In this type of procedure, ophthalmologists must still use forceps with an intermediate pressure to effectively express the glands. Such treatments are also invasive and can be uncomfortable for the patient. Currently, there are no effective “at home” treatments for MGD.

### SUMMARY

A system, method, and apparatus is configured to treat MGD by applying heat and mechanical stimulation of the eyelid, allowing for self-administration by the user at home in a safe and effective manner.

### DESCRIPTION OF DRAWINGS

FIG. 1 illustrates details of the human eye and the ocular surface of the eye.

FIG. 2 illustrates details of the aqueous/muco-aqueous layer that coats the eye.

FIGS. 3A and 3B illustrate details of the lower eyelid and the meibomian glands thereof.

FIGS. 4A-4D illustrate an example configuration of an underlid portion of a system for treating MGD.

FIGS. 5A-5C illustrate an example configuration of a stimulator wand portion of the system for treating MGD.

FIG. 6 is a schematic illustration of an example configuration of the system for treating MGD.

FIG. 7A illustrates the positioning of components of the system for treating MGD during treatment.

FIG. 7B illustrates the radiofrequency (RF) and electromagnetic (EM) functionality implemented via the components of the system during treatment.

FIG. 8 illustrates the re-positioning of the system components during treatment.

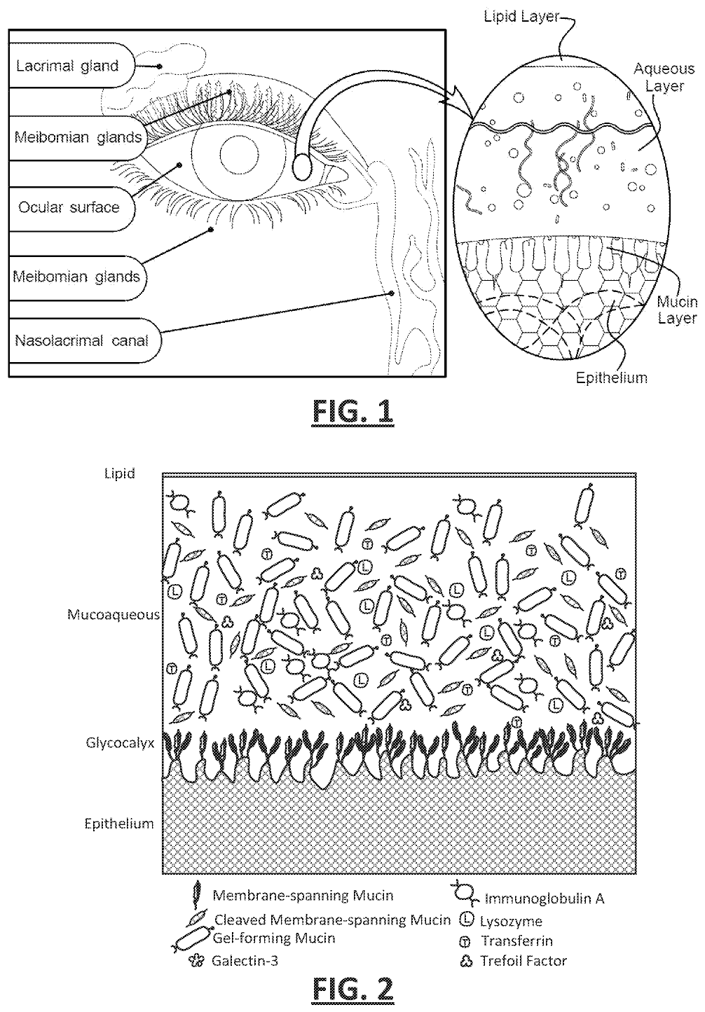

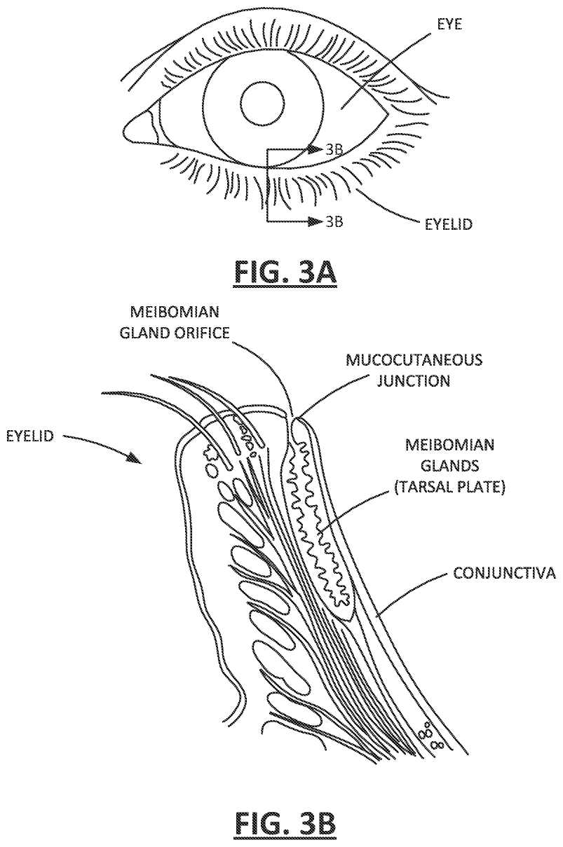

### DESCRIPTION

A system, method, and apparatus is configured to treat MGD by applying heat and mechanical stimulation of the eyelid. The system is configured to allow for self-administration by the user at home in a safe and effective manner. The system 10 includes an under the eyelid (“underlid”) component or device 20 configured to be inserted on the surface of the eye under the eyelid, and an external, hand-held component or device 100, referred to herein as a stimulator wand, configured to be held by the user on or adjacent to the eyelid in the area of the underlid device 20. The stimulator wand 100 generates radio frequency (RF) energy that is used to induce an electric current in the underlid device 20, which is used to excite resistive heating elements that generate heat for heating the meibomian glands. The stimulator wand 100 also generates an oscillating magnetic field that attracts ferromagnetic elements on the underlid device 20 and induces oscillatory movement in the underlid device. The oscillatory movement of the underlid device 20 applies a massaging motion to the meibomian glands while, at the same time, heating the glands via the heating elements.

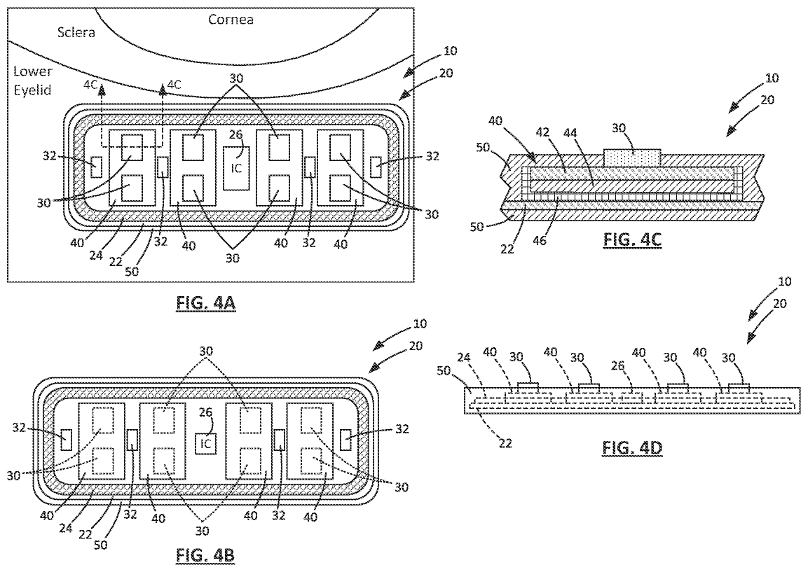

The underlid device 20 that covers the area of the meibomian glands without covering the cornea. As shown in FIGS. 4A-4D, the underlid device can have a shape configured to correspond to the anatomy of the eyelid and the meibomian glands. While the underlid device 20 is shown in FIGS. 4A-4D as having a generally rectangular configuration, other configurations, such as one having a curved edge or surface for following the contour of the eyelid, can be implemented. Covering only the area of the eyelid necessary to treat the meibomian glands avoids the need for anesthetization of the eye surface, which allows the system to be used for self-administered at-home MGD treatment.

Referring to FIGS. 4A-4D, the underlid device 20 includes a substrate 22, such as a flexible printed circuit board (PCB) upon which components are mounted and electrically connected by electrical vias printed/etched on the PCB substrate. The underlid device 20 has a radio frequency (RF) energy harvesting design, including resistive heating elements 30 that are powered via an RF induced electrical current. To facilitate this, the underlid device includes an RF receiving (RX) antenna 24 for energy harvesting. As shown in FIGS. 4A-4D, the RF RX antenna 24 can be a coil that extends around the periphery of the underlid device. The RF RX antenna 24 can, for example, be a wire coil laminated onto the PCB substrate 22, or can be printed/etched onto the PCB substrate.

The underlid device 20 includes an integrated circuit (IC) 26 that is electrically connected to the antenna 24 and to the heating elements 30, e.g., via traces 28. The IC 26 is configured to rectify power harvested via the antenna 24, to distribute power to the heating elements 30, and to control the operation of the heating elements. One or more temperature probes 32 can be distributed on the underlid device 20 and used by the IC 26 to sense the temperature of the device for purposes of safety and control of the heating elements 30. The IC 26 can, for example, utilize a closed-loop control scheme for maintaining a desired temperature of the underlid device 20 using temperature sensed via the probes 32 as a feedback.

As shown in the side view of FIG. 4D, the heating elements 30 are positioned on an eyelid side of the underlid device 20, and are shielded by a thin stack 40 positioned on a sclera side of the device. In the example configuration of FIGS. 4A-4D, there are eight heating elements 30 arranged in groups of two, each of which have an associated thin stack 40. Thus, the example configuration of the underlid device 20 includes four thin stacks 40.

As shown in the zoomed sectional detail of FIG. 4C, each thin stack 40 includes a reflective thermal heat shield 42 positioned adjacent the associated heating element(s). Adjacent to the heat shield 42 (beneath it, as viewed in FIG. 4C) is a ferromagnetic structure 44, which can be constructed of a permanent magnet material or a ferromagnetic material with a high magnetic permeability. The ferromagnetic structure 44 can, for example, be constructed of a mu-metal material, which is a class of highly permeable nickel-iron soft ferromagnetic alloys. Adjacent to the ferromagnetic structure 44 (beneath it, as viewed in FIG. 4C) is a low absorption (k) thermal insulating material 46, such as an aerogel material. The thermal insulating material 46 can also extend around the ends/edges of the heat shield 42 and ferromagnetic structure 44. As shown in

FIG. 4C, the thin stack 40 shields the sclera from the heating elements 30, which act on the eyelid, specifically the meibomian glands.

All of the components of the underlid device—the PCB substrate 22, IC 26, heating elements 30, and the thin stack, are encapsulated in a body of overmolded material 50 that serves as a moisture barrier and provides a biocompatible interface with the eye/eyelid. The body 50 can, for example, be constructed of a silicone hydrogel material. As shown in the side view of FIG. 4D, the body 50 can include recesses or trenches 52 formed around the heating elements 30 on the eyelid side of the device. The recesses/trenches 52, in effect, raise the position of the heating elements 30 so that they project from the eyelid side of the device. This helps increase the local pressure with which the heating elements 30 are held against the eyelid and meibomian glands and therefore improves heat transfer from the heating elements to the eyelid. Additionally or alternatively, features such as dimples and/or chevrons can also be used to achieve this function.

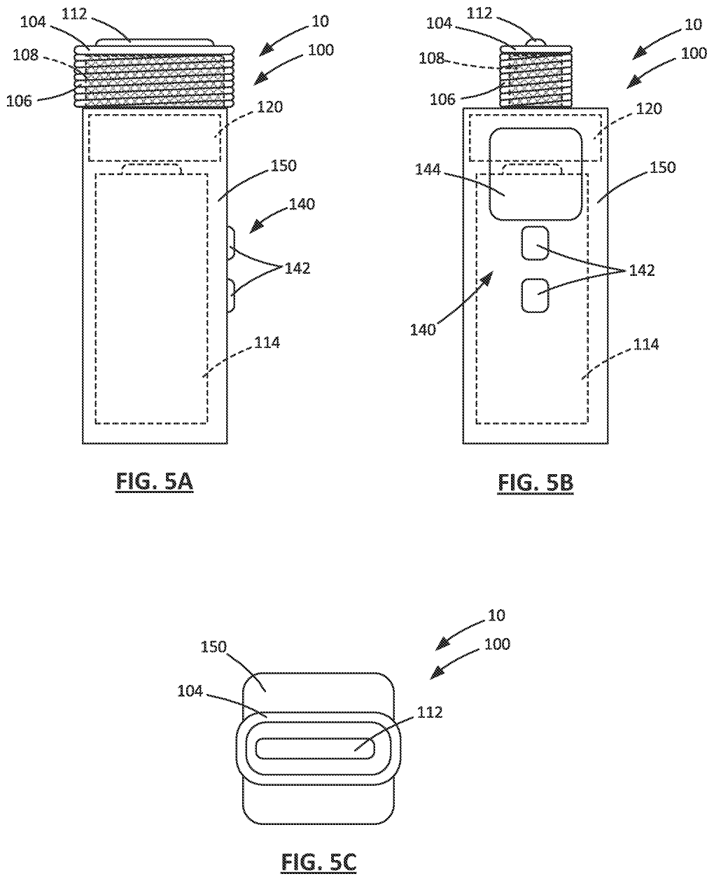

The MGD treatment system 10 also includes a handheld stimulator device 100, referred to herein as a stimulator wand, that is used in conjunction with the underlid device to apply heat and mechanical stimulation to the eyelid. This is shown in FIGS. 5A-5C. As shown in these Figs., the stimulator wand 100 includes an RF transmitting (TX) antenna 102 for power transmission to the underlid device, and for communication with the underlid device. The stimulator wand 100 also includes an electromagnet 104 which, as an example, includes an electromagnetic (EM) coil 106 wrapped around a ferrite core 108. The electromagnet 104 is energizable to generate the magnetic field that attracts the ferromagnetic structure 44 in the thin stack(s) 40 of the underlid device 20 to apply pressure to the eyelid. The RF TX antenna 102 and electromagnet 104 are positioned at an application end 110 of the stimulator wand 100, along with a pressure sensor 112 configured to sense the squeeze pressure applied to the eyelid during use. The end 112 of the stimulator wand 100 that engages the eyelid is formed from or coated with an anti-microbial material.

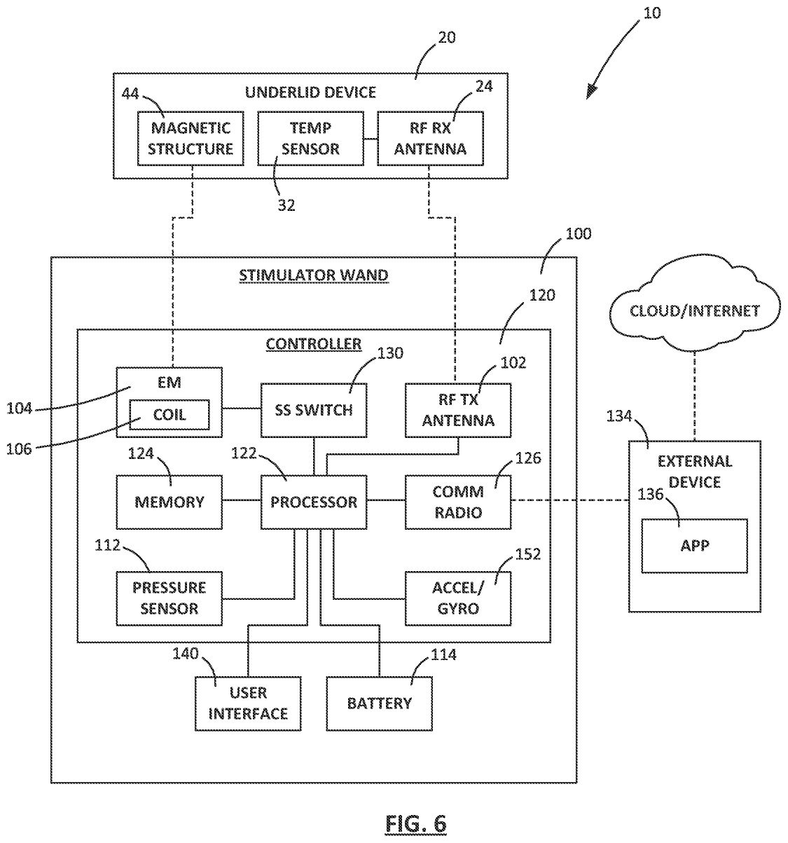

The stimulator wand 100 also includes an electronics module 120 and a battery 114 for powering the electronics module. The electronics module 120 can, for example, include an application-specific integrated circuit (ASIC). The electronics module 120 is illustrated schematically in FIG. 6 and includes a processing component 122, memory devices 124 (RAM/NVRAM), communication radio(s) 126 (e.g., Bluetooth, NFC, Wi-Fi, etc.), and digital, solid-state devices 130 for switching/modulating power to the RF TX antenna 102 in order to induce current in the RF RX antenna 24 of the underlid device 20. The processor 122 is configured to execute a software application that controls the operation of the stimulator wand 100 and its components. The stimulator application, settings, and other data/applications are stored in the memory 124. The communication radio 126 is configured to communicate with a connected device 134 running a custom application 136, which is referred to herein as a smartphone running a custom smartphone app. Other implementations of the connected device 134 and custom application 136 can, of course, be implemented. Examples can include a tablet computer or a PC (desktop or notebook).

The communication radio 126 can also allow for uploading program data, settings, software/firmware updates, and any other data from the smartphone 134 to the stimulator wand 100. Stimulation data can include, for example, stimulation wave forms for the RF TX antenna 102 and parameters/algorithms for modulating the DC signal supplied to the EM coil 106. Similarly, the radio communication can allow for downloading stimulation data from the stimulation wand 100 to the app 136 on the smartphone 134. The downloaded stimulation data can be used to enable tracking or logging of stimulation therapy through a variety of channels, including:
Recording and storing user inputs.
Recording and storing stimulator settings.
Storing safety settings and parameters.
Tracking applied pressure.
Tracking wand motion via onboard accelerometer/gyroscope.
Tracking treatment day/date/time.
Tracking treatment schedule and sending reminders.
Tracking treatment frequency and duration.
Querying and recording effectiveness of treatment.

The smartphone app 136 can utilize internet connectivity to act as an interface between the stimulator wand 100 and the internet/cloud. In doing so, software/firmware updates, stimulator settings, and other data can be downloaded to the smartphone 134 from the internet and then transferred to the stimulator wand 100 from the smartphone. Similarly, stimulator settings, logged stimulator data, and other data can be transferred from the stimulator wand 100 to the smartphone 134, and uploaded from the smartphone to the internet/cloud. This internet connectivity can, for example, allow for a third party, such as a physician, to adjust stimulator settings and review stimulation logs.

The electronics module 120 is operatively connected to the RF TX antenna 102 and to the EM coil 106 so that their respective operations can be controlled. A user interface 140, which can include input buttons 142 and/or knobs, as well as a small screen 144 (LED or LCD, for example). The electronics module 120 controls the operation of the RF antenna 102 and EM coil 106 in response to inputs received via the user interface 140. All components of the stimulator wand 100 are mounted in or on a housing 150 that can be ergonomically designed for handheld use. For reference, the stimulator wand 100 can have a form factor similar in size to that of a whiteboard marker.

Through the user interface 140, the user can control the operation of the stimulator wand 100 according to the stimulation settings stored in memory 124. RF settings (e.g., amplitude, frequency, etc.) define how the RF signal is controlled to produce a desired heating of the underlid device 20. EM settings define how the DC pulse applied to the EM coil 106 produces an EM field to induce a desired pulsating/vibratory motion through magnetic coupling to the ferromagnetic structure 44 of the underlid device 20.

To track treatment, the stimulator wand 100 can include an instrument 152 accelerometer and/or gyroscope for position, attitude, and motion sensing. These components can also be mounted on the ASIC of the electronics module 120. Through this treatment tracking, the system 10 can analyze treatment motions and compare them with the therapeutic results to determine which movements are effective and which are not. Treatment tracking can also monitor the amount of time spent at locations along the eyelid and the pressure applied at those locations to determine whether more treatment time should be spent at certain locations. Through this tracking, the system can learn what treatments methods are most effective and can offer instructions or suggestions to the user accordingly.

At the same time, the system 10 can also track treatment date, time of day, and duration and can send reminders accordingly. The system 10 can also build and maintain a therapy log and use the data stored therein to further refine the treatment regimen. Through this, the system 10 can determine the best thermal stimulation settings for melting the meibum clogging the meibomian glands, and the best mechanical stimulation settings to produce an eyelid squeezing and/or massaging motion for expressing the meibum from the glands.

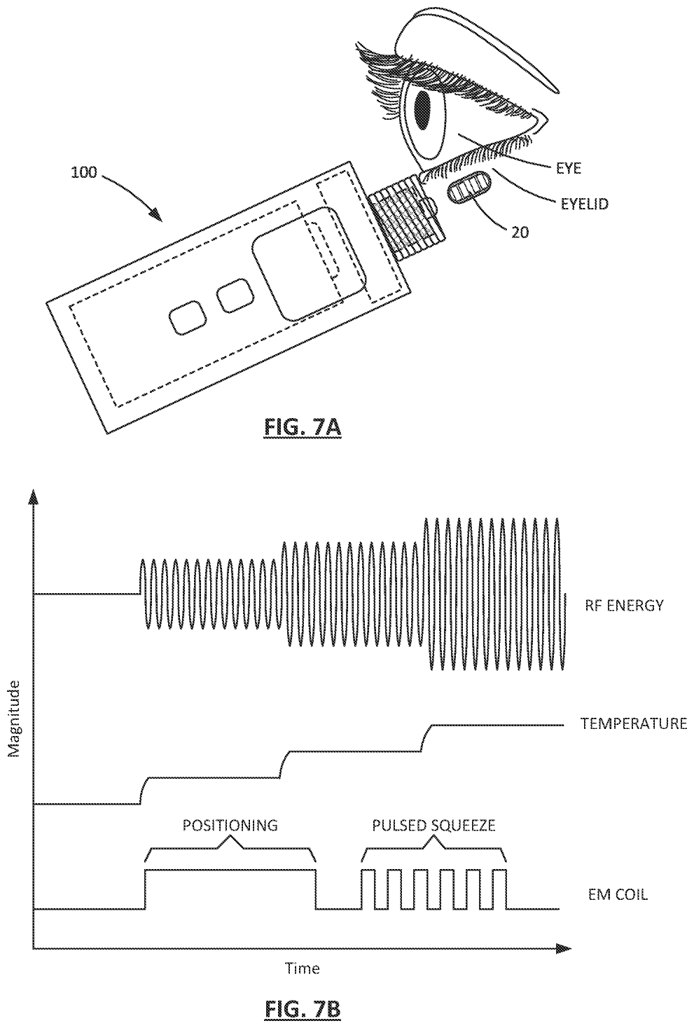

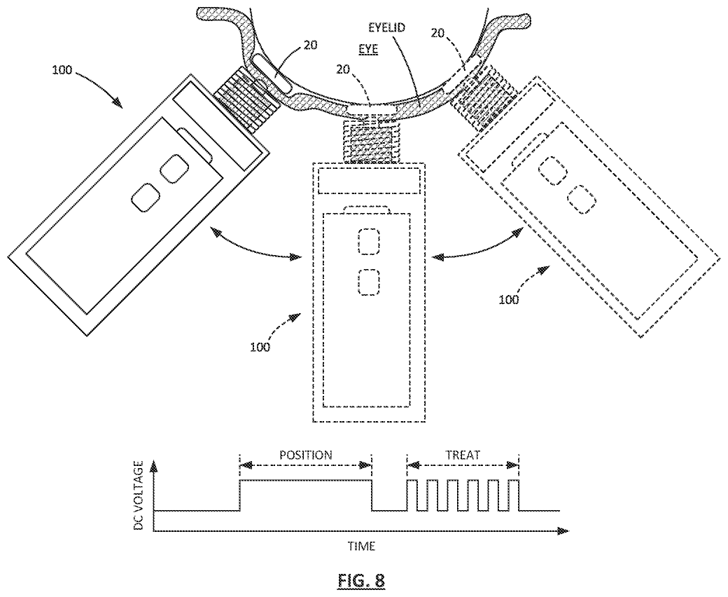

As shown in FIGS. 7A and 7B, the stimulator wand 100, via the RF TX antenna 102 emits an RF signal that excites the RF RX antenna 24 on the underlid device 20. The RF signal emitted from the stimulator wand is rectified and controlled by the control module 120 to energize the heating elements 30 on the underlid device. As shown in FIG. 7B, the RF signal can be modulated to increase/decrease its strength or amplitude which, in turn, can be used to increase/decrease the amount of heat applied by the underlid device and the resulting temperature of the inner eyelid. The temperature is remotely sensed via the temperature sensors 32 and transmitted to the controller 120 which uses the sensed temperature as feedback for controlling the application of heat to the eyelid. The stimulator wand 100 and the underlid device 20 can therefore cooperate to control heat application, to limit the amount of heat applied to the eyelid, and to serve as a safety mechanism for preventing overheating.

At the same time, the electromagnet coil 104 on the stimulator wand 100 is energized, and the coils 106 create a magnetic field that acts on the ferromagnetic core 108 which, in turn, focuses the magnetic field at the end of the stimulator wand. When the stimulator wand 100 is positioned close to the underlid device 20, the magnetic field acts on the magnetic elements 44 in the thin stacks 40 of the underlid device to magnetically attract the underlid device. This magnetic attraction between the stimulator wand 100 and the underlid device 20 creates a mechanical force that exerts pressure on the eyelid, squeezing the eyelid between the stimulator wand and the underlid device. The pressure sensor 112 of the stimulator wand 100 can be configured to sense the applied squeeze pressure.

Referring to FIG. 7B, while the eyelid is squeezed, the position of the stimulator wand 100 can be manipulated by the user to cause a corresponding change in position of the underlid device 20. The user can thus move the underlid device 20 to different positions under the eyelid so that the MGD treatment can be applied over the entire width of the eyelid. The magnetic field itself can also be modulated in order to induce a corresponding movement of the underlid device 20. The magnetic field can, for example, be modulated to cause the underlid device to apply a pulsatory or vibrational movement to the eyelid.

Advantageously, the RF TX signal emitted by the antenna 102 of the stimulator wand 100 is generated via an adjustable alternating current (AC) signal, whereas the magnetic field emitted by the electromagnet 104 of the stimulator wand 100 is generated by a switchable (via SS switches 130) direct current (DC) signal. This allows the two signals emitted by the stimulator wand 100 to coexist without interfering with each other.

FIG. 8 illustrates the operation of the system 10, method, and apparatus 20, 100 to treat MGD by applying resistive heating and magnetic-based mechanical stimulation of the eyelid.

The underlid device 20 is positioned between the eyelid and the eyeball. The stimulator wand 100 is positioned against the exterior surface of the eyelid, and the EM coil 106 is energized to create a magnetic field that acts on the underlid device 20, which draws the underlid device toward the stimulator wand, compressing the eyelid between. The pressure sensor 112 at the tip of the wand 100 senses the pressure applied to the tip, which is representative of the squeeze placed on the eyelid. At the same time, the RF TX antenna 102 is energized to induce a current in the RF RX antenna 24 of the underlid device 20 for powering the heating elements 30. As a result, the eyelid and the meibum in the meibomian glands is heated and squeezed.

As shown in dashed lines in FIG. 8, movement and other manipulations of the stimulator wand 100 magnetically induces corresponding movement of the underlid device 20. This allows the location of the pressure applied to the eyelid to be adjusted, as indicated generally by the arrows in FIG. 8. Lateral positional adjustments allow the therapy to be applied across the width of the eyelid. Vertical positional adjustments moves the pressure in the direction of the length of the meibomian gland ducts, which helps expel the meibum from the glands. The MGD treatment regimen proceeds through this combination of movements.

Additionally, the stimulator wand 100 can be configured to modulate the frequency and/or amplitude of the DC current supplied to the EM coil 106 signal to produce pulsation in the generated magnetic field. This modulation can be configured to induce vibratory movements in the underlid device 20, which produces a corresponding pulsation in the pressure applied to the eyelid and the meibomian gland. This pulsating pressure helps break-up, dislodge, and move the meibum through the meibomian gland ducts, and to expel the meibum from the meibomian glands.

Advantageously, the pressure is applied to the eyelid without placing excess pressure on the eyeball. The pressure sensor 112 at the tip of the stimulator wand 100 senses the pressure applied to the eyelid and can modulate the EM coil signal to control the application of pressure within predetermined bounds, for both treatment efficacy and for safety purposes. By distributing the ferromagnetic material 44 of the thin stacks 40 across the area of the underlid device 20, the pressure can be applied uniformly across the underlid device, providing both repeatable and reliable MGD treatment.

The electromagnet 104 includes a ferromagnetic core 108 and an EM coil 106 wrapped around the core. The configuration of the electromagnet 104 and its components implemented in the stimulator wand 100 can differ in form so as to comply with the configuration and form factor of the stimulator wand, without departing or detracting from the features and function described herein.

The ferromagnetic core 108 can be constructed of a high magnetic permeability material, such as soft iron. The EM coil 106 can be constructed of a relatively thin, e.g., 36 gauge, EM wire. For the above example, assuming a typical 3 mm eyelid thickness, a 1.4 Tesla electromagnet will provide a magnetic squeeze force sufficient to provide the desired pressure between the stimulator wand and the underlid device. To produce this, in an example configuration, the soft iron forming the core can have a relative magnetic permeability (μl μ₀) of 5,000, and the EM coil is formed from 36 gauge EM wire at about 100 turns/inch and 400 ohms/foot resistance.

According to this design, at 100 turns/inch, a 1-inch long electromagnet with a ¼-inch wide head has a length of approximately:

100×2π×¼-inch≈157 inches≈13 feet.

Exciting the EM coil having this configuration at a coil current of 0.2 A leads to a voltage drop of 1 volt and produces an approximately 2.5T magnetic field.

Additionally, the shape of the magnet core 108 produces a magnetic field where the EM field coupling and EM field gradient creates lateral forces on the underlid device to lock its lateral position in place. This can be produced, for example, by configuring the core with converging legs around which the EM coil is wrapped. This configuration shapes the flux field of the electromagnet 104 in a manner that captures and constrains the underlid device. This creates stability in the underlid device 20, as it is constrained from moving out of alignment with the tip of the stimulator wand 100. This is especially beneficial since the intent is to move the wand 100 and to produce a magnetically induced movement of the underlid device 20 while, at the same time, exerting a squeeze pressure on the eyelid.

The following outlines an example implementation of the MGD treatment system 10 as used to treat MGD:
First, the underlid device 20 is inserted in the underlid region of the upper or lower eyelid, as the system 10 can be used to treat MGD in either location.
Next, the tip of the stimulator wand 100 is brought close to the outside surface of the eyelid proximate to the underlid region where the underlid device 20 is positioned.
The user then activates the system through the user interface 140 on the stimulator wand 100, which initiates the transmission of RF energy to apply heat to the lid via the heating elements 30 in the underlid device 20. The temperature sensors 32 in the underlid device 20 will detect temperature on the eyelid surface (heating surface) as well as the eyeball surface and ensure the operation is within the safety range.
Once the sensed temperature reaches a predetermined threshold, the EM coil 106 is energized with a DC current, causing the electromagnet 104 to “grab” onto the ferromagnetic elements 42 of the underlid device 20 and sandwich the eyelid. EM coupling between the stimulator wand 100 and the underlid device 20 can be confirmed by the pressure sensor 112 in the stimulator wand.
With the underlid device 20 coupled to the electromagnet 104 in the stimulator wand 100, the EM current can be modulated to produce pulsation in the squeeze force. The pressure sensor 112 on the tip of the smart wand 100 will ensure no excessive force will be applied to the eyelid due to excessive current driving the electromagnet 104. The pressure sensor 112 can also sense the user inadvertently applying pressure via the manual maneuvering of the stimulator wand 100.
The user utilizes the magnetic coupling between the stimulator wand 100 and the underlid device 20 to slide the underlid device along the eyelid to mechanically stimulate meibomian glands in a “wiping” motion for expressing the meibum from the glands. The underlid device 20 can be moved to another treatment area by repositioning the stimulator wand 100.
Throughout the treatment, utilizing the accelerometer/gyroscope 152 features, the stimulator wand 100 can track the wand motions used to expel the meibum and to keep track of the underlid locations that are treated. The treatment time/duration at each location is recorded, as is the thermal and mechanical characteristics of the applied treatment (wiping pattern, or pulsed action, heat levels, etc.) at each location. All of this recorded data can be transmitted (e.g., via Bluetooth connection) to the external device 134 via the app 136, which can transmit the data to the cloud/internet for remote access and processing. The app 136 can give the user useful feedback on the areas that have been treated and can coach/instruct on where additional treatment should be focused. This feedback/coaching can come from remote processing on the cloud/internet.
The app 136 can send reminders to the user for follow up treatments, and can also send push notifications to ask the user caring questions related to their dry eye disease and treatment regimen such that the treatment becomes proactive instead of reactive.
The system 10 can also use the collected data, along with data related to the user answers regarding treatment queries, so that the system can learn what types of treatment are most effective. The system 10 can then coach/instruct the user on future or additional treatments that can be implemented according to what are determined to be the most effective treatment routines.

What have been described above are examples of the present invention. It is, of course, not possible to describe every conceivable combination of components or methodologies for purposes of describing the present invention, but one of ordinary skill in the art will recognize that many further combinations and permutations of the present invention are possible. Accordingly, the present invention is intended to embrace all such alterations, modifications, and variations that fall within the spirit and scope of the appended claims.

1. A stimulator system for massaging an eyelid to move meibum through a meibomian gland duct toward a gland duct orifice at the eyelid margin in order to clear meibum blockage in the gland duct, comprising:
an underlid device configured to be positioned between the eyelid and the eyeball surface, the underlid device comprising a ferromagnetic element;
a stimulator wand comprising an electromagnet configured to produce an electromagnetic (EM) field;
wherein the stimulator wand is configured to be positioned against an outer surface of the eyelid, and the electromagnet is configured to be energized so that the EM field acts on the ferromagnetic element in the underlid device to magnetically couple the stimulator wand to the underlid device.

2. The stimulator system recited in claim 1, wherein the underlid device further comprises one or more heating elements and an RF receiver antenna for powering the heating elements via an inducted current generated in the RF receiver antenna, wherein the stimulator wand comprises an RF transmitter antenna configured to generate an RF signal that acts on the RF receiver antenna of the underlid device to induce the current in the RF receiver antenna.

3. The stimulator system recited in claim 2, wherein the EM field produced by the electromagnet is oscillating and induces oscillatory movement in the underlid device configured to apply a massaging motion to the meibomian glands while, at the same time, heating the glands via the heating elements.

4. The stimulator system recited in claim 2, wherein the underlid device further comprises an integrated circuit (IC) that is electrically connected to the RF receiver antenna and to the heating elements, the IC being configured to rectify power harvested via the RF receiver antenna, to distribute power to the heating elements, and to control the operation of the heating elements.

5. The stimulator system recited in claim 2, wherein the underlid device further comprises one or more temperature probes distributed on the underlid device and used by the IC to sense the temperature of the device for purposes of safety and control of the heating elements.

6. The stimulator system recited in claim 2, wherein the heating element and ferromagnetic element are arranged in a thin stack on the underlid device, wherein each thin stack comprises a reflective thermal heat shield positioned adjacent the heating element and a low absorption (k) thermal insulating material adjacent the ferromagnetic element.

7. The stimulator system recited in claim 2, wherein the underlid device comprises a body of overmold material that encapsulates the underlid device.

8. The stimulator system recited in claim 7, wherein the body of overmold material comprises a silicone hydrogel material.

9. The stimulator system recited in claim 2, wherein the underlid device comprises recesses formed around the heating elements on the eyelid side of the device, the recesses being configured to effectively raise the position of the heating elements so that they project from the eyelid side of the underlid device to increase the local pressure with which the heating elements are held against the eyelid and meibomian glands to improve heat transfer from the heating elements to the eyelid.

10. The stimulator system recited in claim 2, wherein the RF signal can be modulated to increase/decrease its strength or amplitude in order to produce a corresponding increase/decrease in the amount of heat applied by the underlid device.

11. The stimulator system recited in claim 2, wherein the magnetic attraction between the stimulator wand and the underlid device creates a mechanical force that exerts pressure on the eyelid, squeezing the eyelid between the stimulator wand and the underlid device.

12. The stimulator system recited in claim 2, wherein the stimulator wand includes a pressure sensor configured to sense the applied squeeze pressure.

13. The stimulator system recited in claim 2, wherein the stimulator wand is configured to modulate the magnetic field produced by the electromagnet in order to induce a corresponding pulsatory and/or vibrational movement of the magnetically coupled underlid device.

14. The stimulator system recited in claim 2, wherein the RF signal emitted by the stimulator wand is generated via an adjustable alternating current (AC) signal, and the magnetic field emitted by the stimulator wand is generated via a switchable direct current (DC) signal.

15. The stimulator system recited in claim 2, wherein the electromagnet comprises electromagnetic (EM) coil wrapped around a ferrite core.

16. The stimulator system recited in claim 2, wherein the stimulator wand includes an electronics module comprising an application-specific integrated circuit (ASIC) that includes one or processors, memory devices, communication radios, and digital devices for switching/modulating power to the RF transmitting antenna and electromagnet.

17. The stimulator system recited in claim 16, wherein the processor is configured to execute a software application that controls the operation of the stimulator wand and its components, the stimulator application, settings, and other data/applications being stored in the memory, and the communication radio is configured to communicate with a connected device running a custom application.

18. The stimulator system recited in claim 16, wherein the communication radio is configured to upload program data, settings, software/firmware updates, and any other data from the connected device to the stimulator wand.

19. The stimulator system recited in claim 16, wherein the communication radio is configured to downloading stimulation data from the stimulation wand to the connected device.

20. The stimulator system recited in claim 19, wherein the downloaded data can include user inputs, stimulator settings safety settings and parameters, applied pressure, wand motion, treatment day/date/time, treatment schedule and reminders, treatment frequency and duration, and querying and recording effectiveness of treatment.

21. The stimulator system recited in claim 17, wherein the connected device is configured to utilize internet connectivity to act as an interface between the stimulator wand and the internet, wherein the internet connectivity enables software/firmware updates, stimulator settings, and other data to be downloaded to the connected device from the internet and then uploaded to the stimulator wand from the connected, and wherein the internet connectivity enables stimulator settings, logged stimulator data, and other date to be downloaded from the stimulator wand to the connected device, and uploaded from the connected device to the internet.

22. The stimulator system recited in claim 17, wherein the connected device comprises a smartphone.

23. The stimulator system recited in claim 2, wherein the stimulator wand comprises an onboard accelerometer and/or gyroscope for tracking the position, attitude, and motion of the stimulator wand.

24. The stimulator system recited in claim 16, wherein the stimulator wand comprises a user interface to the electronics module, wherein the electronics module controls the operation of the RF antenna and EM coil in response to inputs received via the user interface.

25. The stimulator system recited in claim 2, wherein the electromagnet comprises a magnet core is configured to produce a magnetic field in which the electromagnetic field coupling and electromagnetic field gradient creates lateral forces on the underlid device to constrain movement of the underlid device.

26. A method for treating meibomian gland dysfunction (MGD), comprising the steps of:
providing the stimulator system recited of claim 2;
inserting the underlid device in the underlid region of the upper or lower eyelid;
positioning the tip of the stimulator wand close to the outside surface of the eyelid proximate to the underlid region where the underlid device is positioned;
activating the stimulator wand to initiate the transmission of RF energy to the heating elements to apply heat to the eyelid via the underlid device;
energizing the EM coil so that the electromagnet attracts the underlid device so that the stimulator wand and the underlid device are magnetically coupled and apply a squeeze pressure to the eyelid between the underlid device and the stimulator wand; and
modulating the EM current to produce pulsation in the squeeze pressure.

27. The method recited in claim 26, further comprising the step of utilizing the magnetic coupling between the stimulator wand and the underlid device to slide the underlid device along the eyelid to mechanically stimulate meibomian glands in a “wiping” motion for expressing the meibum from the glands and/or to move the underlid device to another treatment area by repositioning the stimulator wand.
